(19) United States
(12) Patent Application Publication
(10) Pub. No.: US 2024/0091172 A1
(43) Pub. Date: Mar. 21, 2024

(54) DRUG FOR ANTAGONIZING REPLICATION OF PORCINE REPRODUCTIVE AND RESPIRATORY SYNDROME VIRUS AND APPLICATION THEREOF

(71) Applicant: NANJING AGRICULTURAL UNIVERSITY, Nanjing, Jiangsu (CN)

(72) Inventors: Ping JIANG, Nanjing (CN); Xuewei LIU, Nanjing (CN); Juan BAI, Nanjing (CN); Junren ZHANG, Nanjing (CN); Xianwei WANG, Nanjing (CN); Yufeng LI, Nanjing (CN)

(73) Assignee: NANJING AGRICULTURAL UNIVERSITY, Nanjing, Jiangsu (CN)

(21) Appl. No.: 17/767,616

(22) PCT Filed: Jun. 3, 2020
(86) PCT No.: PCT/CN2020/094179

§ 371 Date: Apr. 8, 2022

(30) Foreign Application Priority Data
Oct. 8, 2019 (CN) 201910951121.7

(51) Int. Cl. A61K 31/12; A61P 31/14

(52) U.S. Cl. CPC A61K 31/12; A61P 31/14

(57) ABSTRACT

A drug for antagonizing the replication of porcine reproductive and respiratory syndrome virus (PRRSV) and an application thereof. By using a viral infection test, a drug which antagonizes PRRSV—xanthohumol—is discovered for the first time from among a natural drug library of 386 plant sources. Xanthohumol can effectively inhibit PRRSV replication on both Marc-145 and PAM cells. Five xanthohumol derivatives having different molecular structures are artificially synthesized, and it is discovered that a derivative Xn-4 has the strongest inhibitory effect on virus replication in vitro. Test results of artificial infection and drug therapy in piglets show that the Xn-4 can effectively inhibit PRRSV viremia, relieve clinical symptoms of infected pigs, and significantly reduce lung inflammation and pathological damage.

### TECHNICAL FIELD

The present invention belongs to the technical field of veterinary prevention and treatment drugs, and in particular relates to a new drug for antagonizing the replication of porcine reproductive and respiratory syndrome virus and an application thereof.

### BACKGROUND

The pathogen of porcine reproductive and respiratory syndrome is porcine reproductive and respiratory syndrome virus (PRRSV), which belongs to the Arteriviridae family and is a single-stranded positive sense RNA virus. The virus infection can cause reproductive disorders in pregnant sows, respiratory disorders in pigs of all ages, immune suppression in pigs, and diseases that promote secondary infections, causing huge economic losses to the domestic and foreign pig breeding industry. Commercial vaccines are not yet ideal. The cross-protection ability of vaccines against strains is not complete, and the process of live virus replication in pigs has the possibility of virulence reversion and recombination with wild viruses to form new strains. Porcine reproductive and respiratory syndrome virus (PRRSV) is an important pathogen that endangers the sustainable and healthy development of the pig industry in the world, and commercial vaccines are not yet ideal. It is necessary to research and develop new antiviral natural drugs.

Natural products have the characteristics such as wide distribution, easy availability, and low toxicity. In addition, natural products, especially traditional Chinese herbal medicines, have become valuable resources for new drug discovery due to their remarkable curative effects in clinical treatment of viral infectious diseases. Many drugs were originally developed from natural products, including the well-known anticancer drug paclitaxel and the antimalarial drug artemisinin. Screening antiviral active compounds from natural products and identifying their antiviral mechanisms will provide a basis for the development and application of antiviral drugs. In this study, xanthohumol was selected from a natural product drug library and found to have the ability to resist PRRSV infection in vitro. In view of this, we conducted piglet treatment experiments and found that xanthohumol has therapeutic effects on PRRSV-infected piglets.

### SUMMARY

An objective of the present invention is to provide an application of xanthohumol and a derivative thereof in the preparation of drugs for preventing and treating porcine reproductive and respiratory syndrome.

Another objective of the present invention is to provide a xanthohumol derivative for antagonizing the replication of porcine reproductive and respiratory syndrome virus, and a preparation method and an application thereof.

The objective of the present invention may be achieved by the following technical solution.

An application of xanthohumol and a derivative thereof in the preparation of drugs for preventing and treating porcine reproductive and respiratory syndrome.

The structure of the xanthohumol is shown in formula (I);

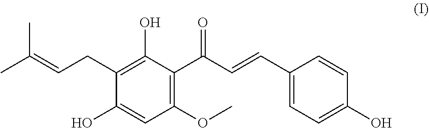 [SMILES: COc1cc(O)c(CC=C(C)C)c(O)c1C(=O)/C=C/c1ccc(O)cc1]

the structure of the xanthohumol derivative is shown in formula (II):

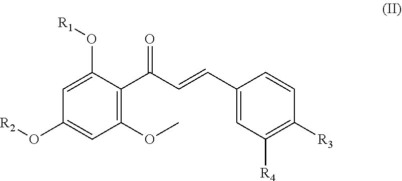

where R₁ is H or CH₃, R₂ is H or CH₃, R₃ is H or OH, and R₄ is H or NO₂.

The most preferred structure of the xanthohumol derivative is shown in formula (III):

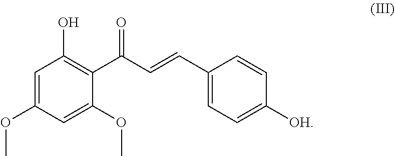 [SMILES: COc1cc(O)c(C(=O)/C=C/c2ccc(O)cc2)c(OC)c1]

A preparation method of the xanthohumol derivative includes the following steps:

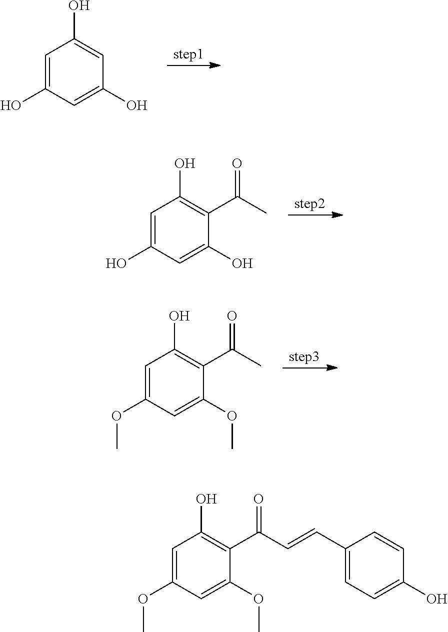 [SMILES: Oc1cc(O)cc(O)c1 ; CC(=O)c1c(O)cc(O)cc1O ; COc1cc(O)c(C(C)=O)c(OC)c1 ; COc1cc(O)c(C(=O)/C=C/c2ccc(O)cc2)c(OC)c1]

(1) Adding m-trisphenol, isopropyl ether, acetonitrile and anhydrous zinc chloride into a reaction vessel, controlling the temperature at 0-5° C., and keeping stirring; introducing dry hydrogen chloride gas until the reaction solution becomes clear from turbidity; continuing to introduce the hydrogen chloride gas for producing a large amount of white solid; after gas introduction, immediately placing the reaction solution in a refrigerator at −10° C. overnight for crystallization; the next day, taking out and filtering the reaction solution, and washing the filter cake with isopropyl ether once; taking out the filter cake, adding water to fully disperse, starting heating, and carrying out atmospheric distillation to remove the residual isopropyl ether from the product; then continuing to heat up to 100° C. to reflux for producing a large amount of white solid, cooling the product to normal temperature, filtering the product, washing the filter cake with water and then drying the filter cake to obtain intermediate 1;
(2) Adding the intermediate 1, acetone, anhydrous potassium carbonate and dimethyl sulfate into the reaction vessel, starting mechanical stirring, heating to reflux until the reaction is complete, filtering the reaction solution, washing the filter cake twice with acetone, concentrating the filtrate to obtain a solid, then washing the solid with petroleum ether and ethyl acetate (V:V) of 10:1, and carrying out filtering and drying to obtain intermediate 2; and
(3) Adding potassium hydroxide to methanol, adding the intermediate 2 after cooling to room temperature, then adding p-hydroxybenzaldehyde, and stirring the mixture until the reaction is complete; adding the reaction solution to water, filtering the reaction solution, taking out the filter cake, adding absolute ethanol, heating the product to reflux, cooling the product to 0° C., and carrying out filtering and drying to obtain the target product.

An application of the xanthohumol derivative in the preparation of drugs for preventing and treating porcine reproductive and respiratory syndrome.

A drug for antagonizing porcine reproductive and respiratory syndrome virus, which contains the xanthohumol or the xanthohumol derivative.

The normal temperature or room temperature as described in the present invention is 25±5° C.

The Present Invention has the Following Beneficial Effects:

By using a viral infection test in this study, a drug which antagonizes PRRSV—xanthohumol (Xn)—is discovered for the first time from among a natural drug library of 386 plant sources. Xanthohumol can effectively inhibit PRRSV replication on both Marc-145 and PAM cells. 5 xanthohumol derivatives having different molecular structures are artificially synthesized, and it is further discovered in a viral infection test that the drug derivative Xn-4 has the strongest inhibitory effect on virus replication in vitro. Test results of artificial infection and drug treatment in piglets show that the Xn-4 can effectively inhibit PRRSV viremia, relieve clinical symptoms of infected pigs, and significantly reduce lung inflammation and pathological damage. Based on the above results, it is proved that the drug has important preventive and therapeutic effects on porcine reproductive and respiratory syndrome, and has important application prospects.

### BRIEF DESCRIPTION OF THE DRAWINGS

FIG. 1 shows a screening process of anti-PRRSV drugs.

FIG. 2 shows the inhibitory effect of Xn on PRRSV replication in Marc145 cells.

FIG. 3 shows the effect of Xn on PRRSV replication in PAM cells.

FIG. 4 shows the effect of Xn on different strains of PRRSV in PAM cells.

FIG. 5 shows the different stages of Xn acting on PRRSV.

FIG. 6 shows the effect of Xn on PRRSV-induced inflammatory reaction in Marc-145 cells.

FIG. 7 shows the effect of Xn on PRRSV-induced inflammatory reaction in PAM cells.

FIG. 8 shows the chemical structures of xanthohumol and analogs thereof.

FIG. 9 shows the NMR identification results of an Xn-1 complex.

FIG. 10 shows the NMR identification results of an Xn-2 complex.

FIG. 11 shows the NMR identification results of an Xn-3 complex.

FIG. 12 shows the NMR identification results of an Xn-4 complex.

FIG. 13 shows the NMR identification results of an Xn-5 complex.

FIG. 14 shows a comparison of the inhibitory effects of different derivatives of xanthohumol on PRRSV replication.

FIG. 15 shows the statistics of clinical symptoms.

FIG. 16 shows the results of viral mRNA qRT-PCR detection in blood, nasal swabs and lung tissue at different times post PRRSV infection.

FIG. 17 shows the general pathological changes and histopathological changes of piglet lungs.

FIG. 18 shows the changes of inflammatory factors in lung tissue.

### DETAILED DESCRIPTION

1 Materials and Method

1.1 Cells and Viruses

Highly pathogenic PRRSV strain BB0907, classic PRRSV strain S1 and NADC30-like strains FJ1402 and Marc-145 cells were preserved in a laboratory; porcine alveolar macrophages (PAM) were isolated from PRRSV, PCV2 and PRV negative piglets at 4-6 weeks of age. The PRRSV strain BB0907, the classic PRRSV strain S1 and the NADC30-like strains FJ1402 and Marc-145 cells involved in the experiment are conventional strains disclosed in the prior art, and the isolation method of the porcine alveolar macrophages (PAM) is also a conventional method disclosed in the prior art.

1.2 Main Reagents

The xanthohumol used for in vitro experiments was purchased from Selleck Chemicals, with a purity >99%. The xanthohumol used for in vivo experiments was purchased from Chengdu Herbpurify Co., Ltd., with a purity >98%. Protoporphyrin IX cobalt chloride (CoPP), an inducer of HMOX1 expression, was purchased from Sigma. An enhanced CCK8 kit was purchased from Beyotime Biotechnology Co., Ltd. An anti-PRRSV N protein antibody (2H7) was prepared and stored by graduate students in our laboratory using conventional methods disclosed in the prior art. A labeled Alex 594 goat anti-mouse fluorescent secondary antibody was purchased from Beyotime Biotechnology Co., Ltd. Reverse transcriptase HiScript II 1st Strand cDNA Synthesis Kit and AceQ® qPCR SYBR® Green Master Mix were purchased from Nanjing Vazyme Biotech Co., Ltd. Dimethyl sulfoxide (DMSO) was purchased from Sigma. Other conventional reagents were of analytical grade.

1.3 Determination of CC₅₀

Marc-145 cells were cultured with 10% (volume ratio) FBS-DMEM containing Xn (1-250 μM) of different concentrations, and incubated at 37° C. for 48 h. Cell viability was detected with the enhanced CCK8 kit. The concentration (CC₅₀) at which Xn was cytotoxic to 50% of the cells was calculated using GraphPad Prism 7.0 software. DMSO was used as a negative control.

1.4 Determination of IC₅₀

Xn of different concentrations was added to a PAMs medium (1-30 μM), and a DMSO negative control group was set up. Cells were inoculated with PRRSV (0.01 MOI) and incubated at 37° C. for 30 h, and the cell supernatant was pipetted and discarded. The cells were fixed with 4% (volume ratio) paraformaldehyde for 15 min, permeabilized with 0.1% (volume ratio) Triton X-100 for 30 min, and washed 3 times with PBS for 3 min each time. A PRRSV N protein monoclonal antibody (2H7) at a dilution of 1:1000 was added, and the cells were incubated at 37° C. for 1 h, and washed 3 times with PBST for 3 min each time. Alexa Fluor 594 goat anti-mouse IgG at a dilution of 1:1000 was added, and the cells were incubated at 37° C. for 45 min. The nuclei were stained with DAPI for 10 min at room temperature. Three photos were recorded randomly with a Nikon A1 confocal microscope. The fluorescence intensity was measured by ImageJ software. Finally, the 50% inhibitory concentration (IC₅₀) of Xn against PRRSV was calculated by GraphPad Prism 7.0 software, and the results were recorded.

1.5 Determination of Virus Titer

Marc-145 cells were cultured in a 96-well plate. When the cell confluence reached 70%, a nutrient solution was pipetted and discarded, and the cell surface was washed 3 times with PBS. Ten-fold serially diluted PRRSV was added to the cells and the cells were incubated at 37° C. for 1 h. The medium was replaced with fresh 2% FBS-DMEM. Virus titers were determined 5 days post-inoculation (dpi) using end-point dilution analysis. The 50% cell infectious dose (TCID50) was calculated by the Reed-Muench method.

1.6 Stage Experiment of Drug

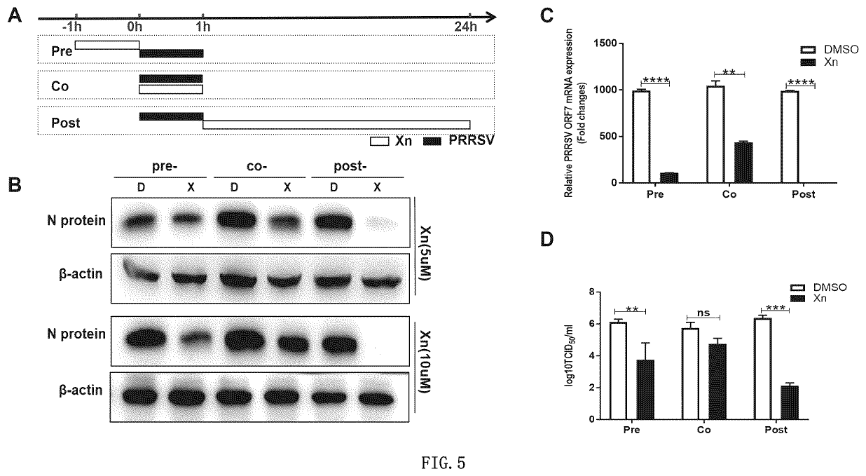

Xn treatment time and PRRSV infection time are shown on the timeline in FIG. 5A. PAMs were inoculated into a 24-well plate, and after complete sedimentation, the time was set as −1 h. 10 μM Xn was added to a pretreatment group (Pre) at −1 h; and at 0 h; cells were inoculated 0.1 moi PRRSV and incubated at 37° C. for 1 h; the virus-containing cell culture was replaced with 2% FBS-DMEM; and the cells were cultured at 37° C. for 24 h. Xn was added to the cells in a co-treatment group (Co) at 0 h; the cells were inoculated with 0.1 moi PRRSV, and incubated at 37° C. for 1 h; the virus-containing cell culture was replaced with 2% FBS-DMEM; and the cells were cultured at 37° C. for 24 h. A post-treatment group (Post) was inoculated with 0.1 moi PRRSV at 0 h; the culture was discarded at 1 h and replaced with 2% FBS-DMEM containing 10 μM Xn; and the cells were cultured at 37° C. for 24 h.

1.7 Experimental Design for Piglet Treatment

25 15-week-old commercial piglets (purchased from a farmer in Nantong, Jiangsu) were detected by ELISA and PCR respectively, and were negative in antibodies and antigens against PRRSV, PCV2, PRV and CSFV. The piglets were randomly divided into 5 groups (5 in each group), the first group was a PRRSV infection and treatment group with normal saline+0.5% (volume ratio) DMSO (positive control group); the second group was a PRRSV infection and treatment group with Xn-4 (5 mg/kg); the third group was a PRRSV infection and treatment group with Xn-4 (10 mg/kg); the fourth group was a PRRSV infection and treatment group with Xn-4 (20 mg/kg); and the fifth group was an uninfected and untreated negative control group. The piglets in the infection groups were injected intranasally and intramuscularly with 1 mL of PRRSV BB0907 strain (3*10⁵ TCID50) respectively. 24 h post infection, the piglets in the infection groups were intramuscularly injected with 5 mg/kg, 10 mg/kg, 20 mg/kg Xn-4 or 0.5% DMSO. Treatments were performed every 3 days until day 14. After infection, the health status and rectal temperature of the piglets were monitored daily. Serum samples and nasal swabs were collected on days 1, 4, 7, 10, and 14 post-challenge. All piglets were killed and dissected on day 14, and serum and lung tissue were collected for viral load determination and histopathological examination.

1.8 Real Time Fluorescence Quantitative PCR

1.8.1 Relative Fluorescence Quantitative Detection of Relative Levels of Gene mRNAs in Cells

Cellular RNAs were extracted following the operation instructions of an OMEGA RNA virus extraction kit. After reverse transcription into cDNA, RNAs were extracted according to the operation instructions of the OMEGA RNA virus extraction kit. Relative fluorescence quantification was performed after reverse transcription, and the primer sequences used were as follows:

mGAPDH-Fwd
5′ CCTTCCGTGTCCCTACTGCCAA 3′
mGAPDH-Rev
5′ GACGCCTGCTTCACCACCTTCT 3′
PRRSV-ORF7-Fwd
5′ AAACCAGTCCAGAGGCAAG 3′
PRRSV-ORF7-Rev
5′ TCAGTCGCAAGAGGGAAAT 3′
mIL-1β-Fwd
5′ TCCCACGAGCACTACAACGA 3′
mIL-1β-Rev
5′ CTTAGCTTCTCCATGGCTACA 3′
mIL-6-Fwd
5′ GCTGCAGGCACAGAACCA 3′
mIL-6-Rev
5′ AAAGCTGCGCAGGATGAG 3′
mIL-8-Fwd
5′ CTGGCGGTGGCTCTCTTG 3′
mIL-8-Rev
5′ CCTTGGCAAAACTGCACC 3′
mTNF-α-Fwd
5′ TCCTCAGCCTCTTCTCCTTCC 3′
mTNF-α-Rev
5′ ACTCCAAAGTGCAGCAGACA 3′

According to the method of 2-ΔΔCt, the relative content of mRNA of each gene in a sample was calculated with a CT value.

1.8.2 Absolute Fluorescence Quantitative Detection of PRRSV RNA Content in Lung, Serum and Nasal Swabs

About 1 g of lung tissue was homogenized in 3 mL of PBS. After three freeze-thaw cycles, the tissue homogenate was centrifuged at 5000 rpm for 10 min to collect tissue homogenate supernatant. The collected blood was centrifuged at 5000 rpm for 5 min to collect serum. Nasal swabs were rinsed in 1 ml of PBS for 2 min, and the nasal swab dilution was centrifuged at 5000 rpm for 10 min and collected. RNAs were extracted according to the operation instructions of the OMEGA RNA virus extraction kit. 2 μL of 5*qRTSuperMix was added to each 8 μL of the extracted RNA sample (10 μL reaction system), and after thorough mixing, a reverse transcription reaction was performed on a PCR machine. The reaction was performed at 25° C. for 10 min, at 50° C. for 30 min, and at 85° C. for 5 min. The product was frozen at −70° C. and stored for later use. Using the above cDNA as a template, the content of PRRSV nucleic acid in the serum, the lung tissue and the nasal swabs was detected by SYBR Green real time PCR. The primer sequences were F: 5′-AATAACAACGGCAAGCAGCAG-3′, and R: 5′-CCTCTGGACTGGTTTTGTTGG-3′. The reaction system contained 10 μL of 2×Power SYBR Green PCR Master Mix (ABI Company), and 2 μL of cDNA, and the concentration of primers F/R was 400 nmol/L. Reactions were performed on an ABI 7300 real time PCR machine. The reaction program was: pre-denaturation at 95° C. for 2 min, at 95° C. for 15 s, and at 61° C. for 31 min, for a total of 40 cycles. A standard curve was constructed using a recombinant plasmid containing the PRRSV ORF7 gene (see Liu Xing's doctoral dissertation from Nanjing Agricultural University, Analysis of the Immunosuppressive Function Gene of Porcine Reproductive and Respiratory Syndrome Virus and Research on the Immunological Characteristics of GPS Glycosylation Site Deleted Virus). Then, using the CT value, the PRRSV virus content in the lung tissue, the serum and the nasal swabs was calculated based on the standard curve.

1.9 Observation of Clinical Symptoms and Pathological Observation

1.9.1 Observation and Statistics of Clinical Symptoms

The rectal temperature of the piglets was measured every day post the challenge. Clinical symptoms, including coat, skin, feeding condition, diarrhea and mental state, were observed. The body weights of the piglets were measured before and after the challenge. The statistical method of clinical symptoms is as follows: according to clinical manifestations (mental state, coat, and diarrhea), breathing and coughing conditions, the three indicators are scored respectively, and the score is in a range of 1-4 points. The more severe the symptoms, the higher the score. 1 point means normal, 2 points means mild symptoms, 3 points means severe symptoms, and all indicators of dead pigs are 4 points. The piglets in all groups were scored every day post the challenge, and the sum of the scores of the three indicators was the clinical score of each pig every day. Data analysis was performed according to the statistical results 14 days post the challenge.

1.9.2 Gross Lesions in Organs

All the piglets were killed and dissected 14 days post the challenge, and the pathological changes in the lungs of each piglet were observed. Edema, interstitial widening and gross lesions were observed and counted.

1.9.3 Preparation and Observation of Pathological Sections

Tissue blocks not larger than 4 mm×2 cm² were collected from porcine lung tissue, fixed with 4% paraformaldehyde, dehydrated in a graded ethanol series, cleared with xylene, embedded in paraffin, trimmed, sectioned, and stained with hematoxylin and eosin. Lung tissue sections were observed under a microscope, and the pathological scoring rules were: 0-no lesions; 1-mild, focal multifocal interstitial pneumonia (<50%); 2-moderate, multifocal consolidation (50-75%); 3-severe, extensive patchy consolidation (75-90%); and 4-severe diffuse (>90%). Statistical analysis of data was performed with GraphPad 7 software, and the differences among groups were compared. P<0.05 indicates significant difference (* indicates P<0.05).

2. Result

2.1 Natural Product Drug Library Screening

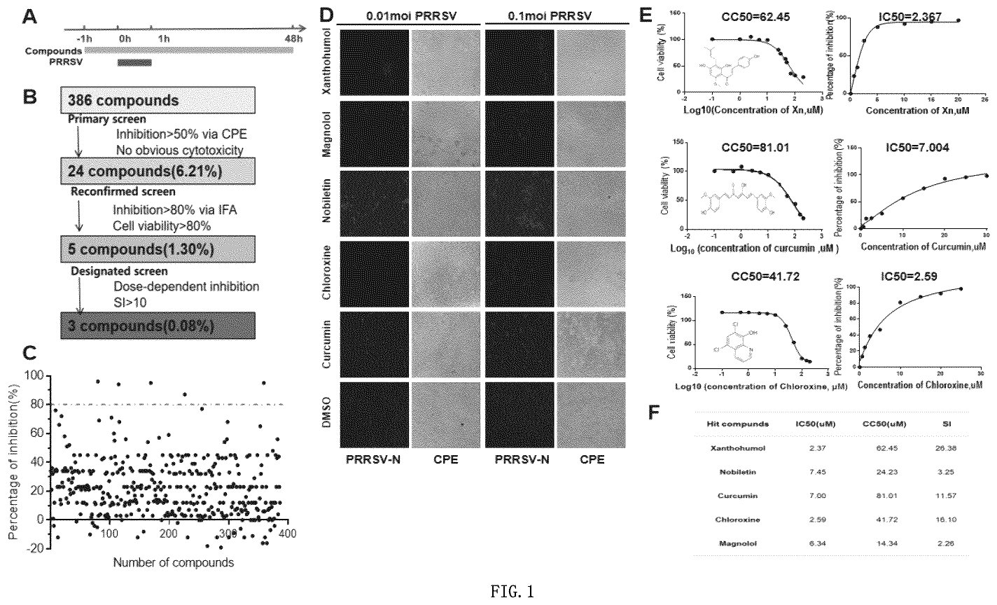

Marc-145 cells were treated with 10 μM of natural products and then infected with PRRSV. In the first round of screening, 24 compounds (6.21%) had no obvious cytotoxicity to Marc-145 cells and could reduce PRRSV-induced CPE by 50% (FIG. 1A). The compounds were subjected to a second round of screening (FIG. 1B), and 5 compounds were found to be less cytotoxic to cells and inhibit PRRSV infection by 80% or more (FIG. 1C and FIG. 1D). Among the 5 compounds, only xanthohumol (Xn), curcumin and chloroxine exhibited dose-dependent inhibitory effects on PRRSV with their selectivity index SI>10 (FIG. 1E and FIG. 1F), and Xn has the lowest IC₅₀, the highest CC₅₀, and the highest selectivity index.

2.2 Xn Inhibits PRRSV in Marc145 Cells.

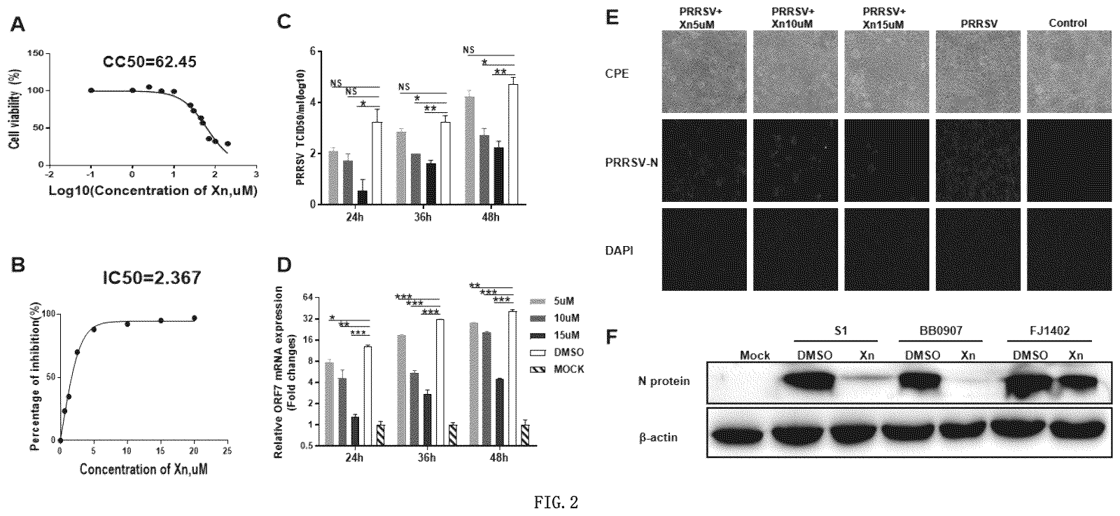

First the cytotoxicity of Xn to Marc145 cells was determined by CCK8 cytotoxicity assay, and it was found that Xn only produced 50% cytotoxicity (CC₅₀ value) at a high concentration (62.45 μM) (FIG. 2A). Further, the inhibitory efficiency of Xn on PRRSV in Marc145 cells was detected by indirect immunofluorescence. As shown in FIG. 2B, Xn produced 50% inhibitory efficiency (IC₅₀ value) on PRRSV at a concentration of 2.367 μM. To further determine the dose range of Xn with the anti-PRRSV activity, Marc-145 cells were treated with 5 μM, 10 μM, and 15 μM Xn respectively, and then infected with PRRSV (0.01 MOI). TCID₅₀ of PRRSV and the ORF7 mRNA level were detected at 24 hpi, 36 hpi and 48 hpi, and the results showed a dose-dependent decrease in viral titers and the ORF7 mRNA level at each time point (FIG. 2C and FIG. 2D). At 48 hpi, both CPE and IFA showed that the number of infected cells in the Xn-treated group was significantly less than that in the negative control group (FIG. 2E). In addition, Western blot analysis showed that Xn could effectively reduce the replication of the three PRRSV strains (FIG. 2F).

2.3 Effects of Xanthohumol in PAM Cells

2.3.1 Xanthohumol Inhibits PRRSV Replication in PAM Cells.

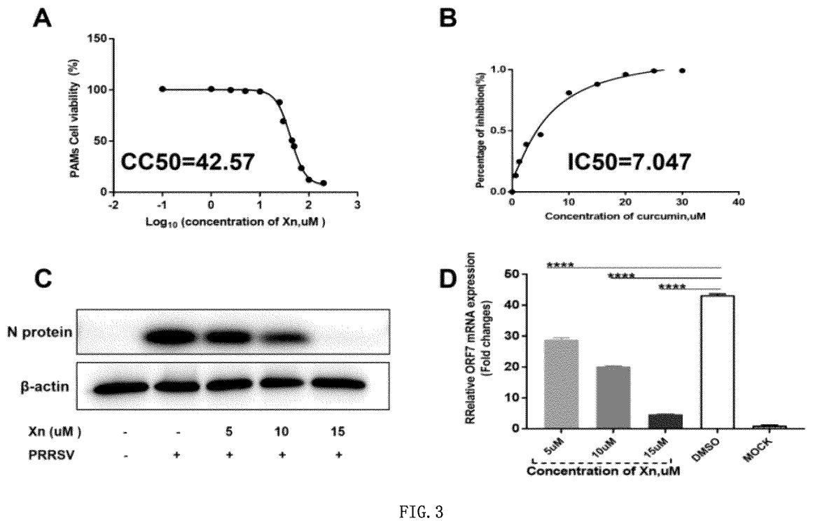

In PAMs cells, Xn had CC₅₀ and IC₅₀ of 42.57 μM and 7.047 respectively (FIG. 3A and FIG. 3B), and decreased the levels of PRRSV N protein and viral mRNA in PAM cells in a dose-dependent manner (FIGS. 3C-D).

2.3.2 Xanthohumol Inhibits Different Subtypes of PRRSV Strains in PAM Cells.

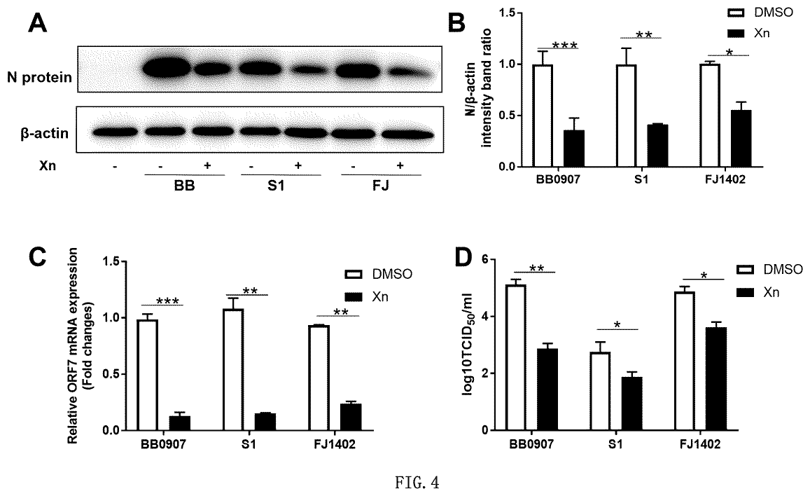

The results are shown in FIGS. 4A-D. In PAM, Xn could inhibit the replication of different genetic subtypes of PRRSV strains in the viral ORF7 gene and protein levels, and reduce the viral infection titer.

2.3.3 Xanthohumol Shows a Therapeutic Effect on PRRSV Infection in PAM Cells.

PAMs were treated with Xn before, during and after PRRSV infection (FIG. 5A). Cell samples were collected at 24 hpi for detection of PRRSV N protein and PRRSV ORF7 mRNA by Western blot and qRT-PCR respectively, and the cell supernatant was collected for virus titer determination. The results showed that Xn treatment post virus infection significantly reduced the level of PRRSV replication, while the pretreatment and co-treatment groups inhibited the virus to a lower degree (FIG. 5B-FIG. 5D), indicating that Xn has a therapeutic effect on PRRSV infection.

2.4 Xn Inhibits PRRSV-Induced Inflammatory Reaction.

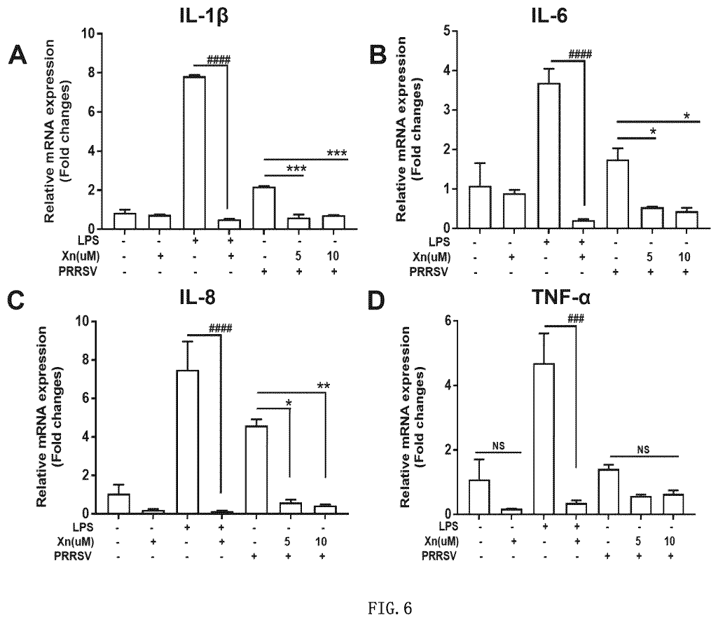

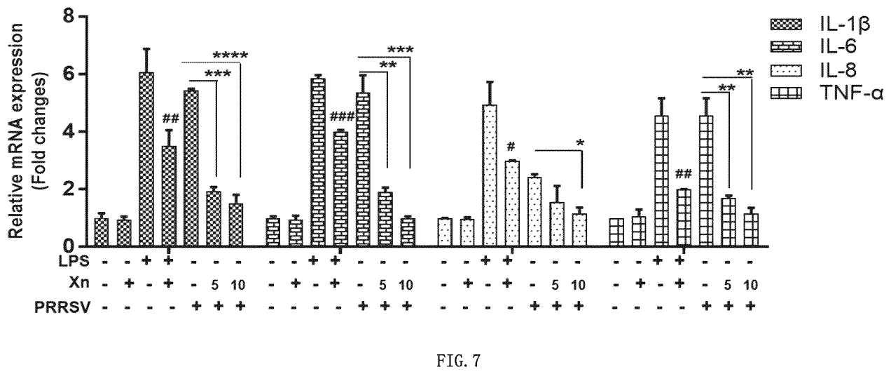

Marc-145 cells and PAM cells were inoculated with 0.1 MOI PRRSV and incubated at 37° C. for 1 h. The nutrient solution was replaced with DMEM-2% FBS containing Xn (or DMSO), and 24 h post infection, cellular RNAs were extracted. A positive control group was set up: Marc-145 cells and PAMs were treated with 10 μg/mL LPS (or DMEM) for 1 h; the cell surface was washed 3 times with PBS; 2% FBS-DMEM containing Xn (or DMSO) was added; and after 6 h, the cells were collected, RNAs were extracted, and the mRNA levels of IL-1β, IL-6, IL-8 and TNF-α were detected by qRT-PCR. The results are shown in FIG. 6 (Marc-145) and FIG. 7 (PAM cells). Both LPS and PRRSV infection could significantly induce increase in the mRNA levels of IL-1β, IL-6, IL-8 and TNF-α. Xn control treatment did not cause changes in intracellular inflammatory factors, but Xn treatment could effectively reduce the mRNA expressions of IL-1β, IL-6, IL-8 and TNF-α induced by LPS and PRRSV in a dose-dependent manner.

2.5 Synthesis and Identification of Different Derivatives of Xanthohumol

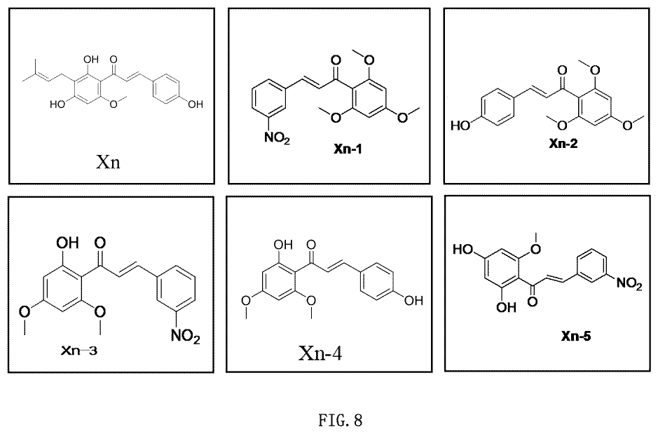

Based on the chemical structure of Xn, five xanthohumol analogs were synthesized, which have the structures shown in FIG. 8, and are named “Xn-1, Xn-2, Xn-3, Xn-4 and Xn-5” respectively, with a purity 90% or more.

2.5.1 Synthesis and Identification of Xn-1 Compound

2.5.1.1 Synthesis of Xn-1 Compound

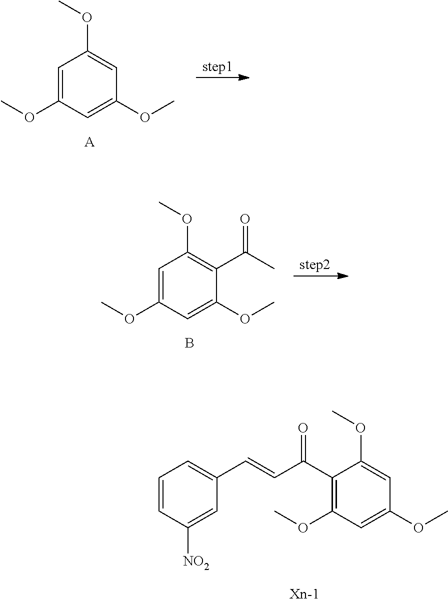

Step I:

Chemical components: 8.4 g (0.05 mol) of 1,3,5-trimethoxybenzene (compound A), 7.6 g (0.075 mol) of acetic anhydride, 30 ml of ethyl acetate, and 4.3 g (0.025 mol) of boron trifluoride ether.

Operation: 8.4 g of 1,3,5-trimethoxybenzene, 7.6 g of acetic anhydride and 30 ml of ethyl acetate were added into a 100 ml reaction flask, the temperature was controlled to be at 10° C., and 4.3 g of boron trifluoride ether was added dropwise. After the dropwise addition, the temperature was raised to 18° C., and the reaction was performed at room temperature for 2 h. TLC monitoring was performed, and after the reaction was complete, the reaction solution was washed with 100 mL of water, 100 ml of 5% (w/w) aqueous sodium bicarbonate solution and 100 ml of saturated brine sequentially. The organic layer was dried by adding 20 g of sodium sulfate for 2 h and filtered, and the filtrate was concentrated to dryness to obtain 8 g of crude product, which was then recrystallized with 40 ml of absolute ethanol to obtain 5.7 g of product (compound B).

Step II:

Chemical components: 10 g (0.047 mol) of compound B, 5.7 g of potassium hydroxide, 60 ml of methanol, and 8.9 g (0.052 mol) of m-nitrobenzaldehyde.

Operation: 5.7 g of potassium hydroxide was added to 60 ml of methanol, the reaction solution was cooled to 25° C. at room temperature, and 10 g of compound B and 8.9 g of compound m-nitrobenzaldehyde were added sequentially. The reaction was performed at room temperature for 24 h. After TLC detected that the reaction was complete, the reaction solution was added to 400 ml of water and filtered. The filter cake was taken out to reflux for 1 h by adding 100 ml of anhydrous ethanol, the reaction solution was cooled to 0° C., allowed to stand for 1 h and filtered, and the filter cake was dried to obtain 15 g of product (Xn-1).

2.5.1.2 Identification Results of Xn-1 Complex

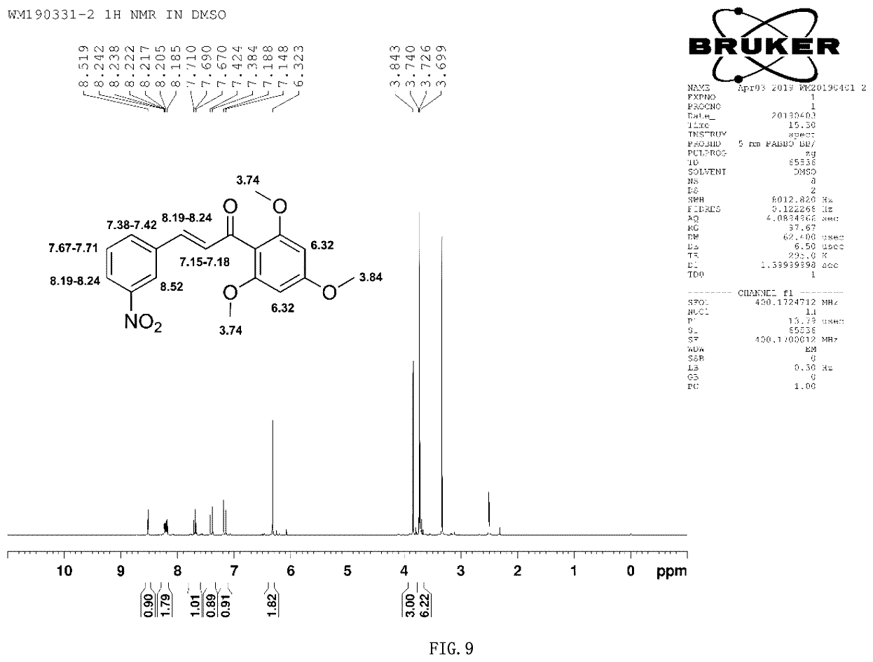

Identification was performed by an NMR method, and the results are shown in FIG. 9. The hydrogen spectrum data are as follows: 3.74 (s, 6H), 3.84 (s, 3H), 6.32 (s, 2H), 7.15-7.18 (d, 1H), 7.38-7.42 (d, 1H), 7.67-7.71 (t, 1H), 8.19-8.24 (dd, 2H), and 8.52 (s, 1H), which meet the structural requirements of the molecular design.

2.5.2 Synthesis and Identification of Xn-2 Compound

2.5.2.1 Synthesis of Xn-2 Compound

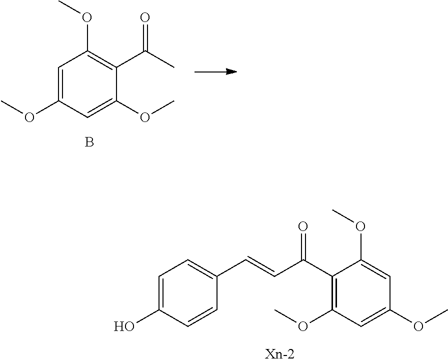

Chemical components: 10 g of compound B, 5.7 g of potassium hydroxide, 60 ml of methanol, and 7.4 g of p-hydroxybenzaldehyde.

Operation: 5.7 g of potassium hydroxide was added to 60 ml of methanol, the reaction solution was cooled to 25° C., and 10 g of compound B and 7.4 g of p-hydroxybenzaldehyde were added sequentially. The reaction was performed at room temperature for 24 h. After TLC detected that the reaction was complete, the reaction solution was added to 400 ml of water, fully dispersed and filtered. The filter cake was taken out to reflux for 1 h with 100 ml of anhydrous ethanol, the reaction solution was cooled to 0° C., allowed to stand for 1 h and filtered, and the filter cake was dried to obtain 10 g of product (Xn-2).

2.5.2.2 Identification Results of Xn-2 Complex

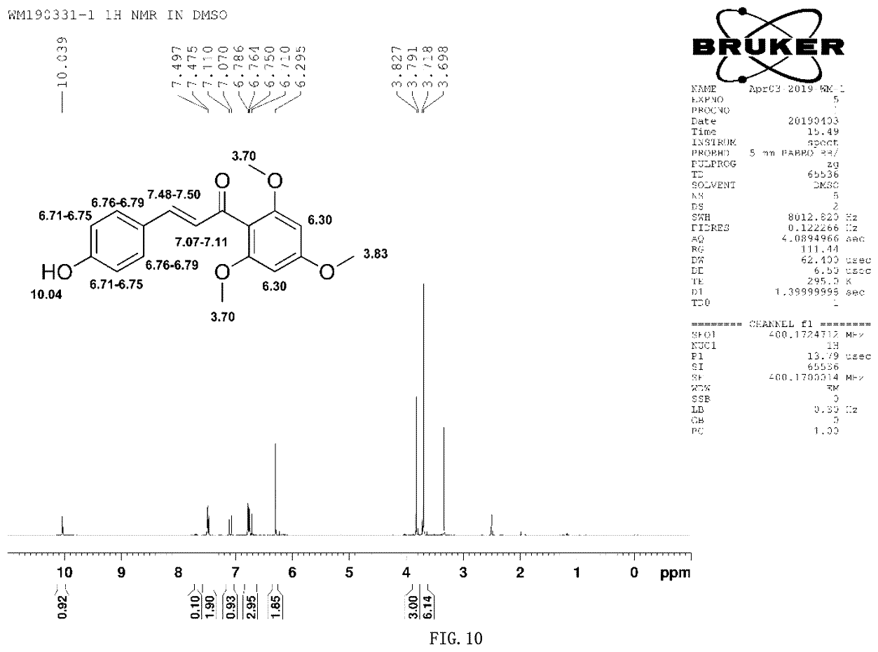

Identification was performed by an NMR method, and the results are shown in FIG. 10. The hydrogen spectrum data are as follows: 3.70 (s, 6H), 3.83 (s, 3H), 6.30 (s, 2H), 6.71-6.75 (d, 2H), 6.76-6.79 (d, 2H), 7.07-7.11 (d, 1H), 7.48-7.50 (d, 1H), and 10.04 (s, 1H), which meet the structural requirements of the molecular design.

2.5.3 Synthesis and Identification of Xn-3 Compound

2.5.3.1 Synthesis of Xn-3 Compound

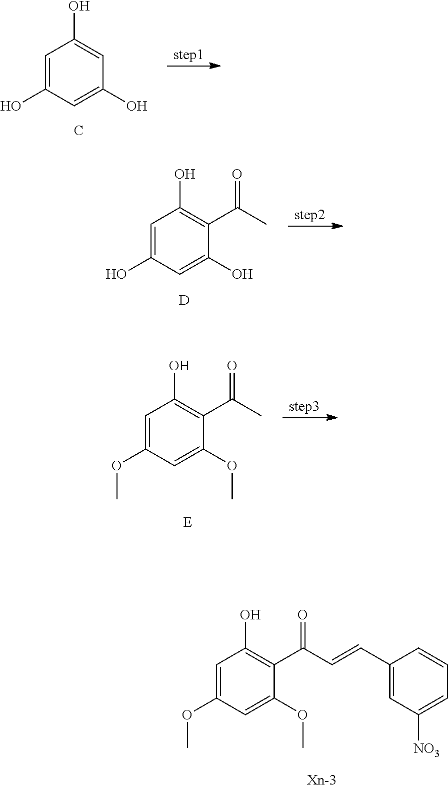

Step I:

Chemical components: 75 g of m-trisphenol (compound C), 108 ml of acetonitrile, 250 ml of isopropyl ether, 15 g of anhydrous zinc chloride, 400 ml of concentrated sulfuric acid, 300 ml of hydrochloric acid, and 200 g of sodium chloride.

Operation: 75 g of m-trisphenol, 250 ml of isopropyl ether, 108 ml of acetonitrile and 15 g of anhydrous zinc chloride were added into a 500 ml four-neck flask, and the temperature was controlled at 0-5° C. Dry hydrogen chloride gas (prepared by adding concentrated sulfuric acid dropwise to a sodium chloride hydrochloric acid solution) was introduced, and the solution was turbid at first, and gradually became clear with the introduction of the hydrogen chloride. With continuous introduction of the gas, a white solid appeared. After the gas was introduced for about 6 h, a large amount of white solid was produced and then the reaction solution was put in a refrigerator overnight (−10° C.). The solid was filtered out the next day, and the filter cake was washed once with 200 ml of isopropyl ether, and then transferred to a 500 ml three-necked flask. 200 ml of water was added, heated and distilled at atmospheric pressure to distill off a small amount of isopropyl ether from the product. Then the temperature was raised to 100° C. to reflux for 5 h to produce a large amount of white solid, the reaction solution was cooled to normal temperature and filtered, and the filter cake was washed with 100 ml of water once and dried to obtain 90 g of product D.

Step II:

Chemical components: 10 g of compound D, 100 ml of acetone, 40 g of anhydrous potassium carbonate, and 25 g of dimethyl sulfate.

Operation: 10 g of compound D, 100 ml of acetone, 40 g of anhydrous potassium carbonate and 25 g of dimethyl sulfate were added into a 250 ml of three-necked flask sequentially, and heated and stirred to reflux for 3 h. When no raw material was detected by TLC, the reaction solution was filtered, the filter cake was washed twice with 50 ml of acetone, the filtrate was concentrated to obtain a solid, and then the solid was washed with petroleum ether and ethyl acetate (V:V) of 10:1 to obtain 8 g of product E.

Step III:

Chemical components: 10 g of compound E, 5.7 g of potassium hydroxide, 60 ml of methanol, and 9.2 g of m-nitrobenzaldehyde.

Operation: 5.7 g of potassium hydroxide was added to 60 ml of methanol, the reaction solution was cooled to 25° C., and 10 g of compound E and 9.2 g of m-nitrobenzaldehyde were added sequentially. The reaction was performed at room temperature for 24 h. After TLC detected that the reaction was complete, the reaction solution was added to 400 ml of water, fully dispersed and filtered. The filter cake was taken out, 100 ml of anhydrous ethanol was added, the mixed solution was heated and stirred to reflux for 1 h, and then heating and stirring were stopped. The reaction solution was cooled to 0° C., allowed to stand for 1 h and filtered, and the filter cake was dried to obtain about 14 g of product Xn-3.

2.5.3.2 Identification Results of Xn-3 Complex

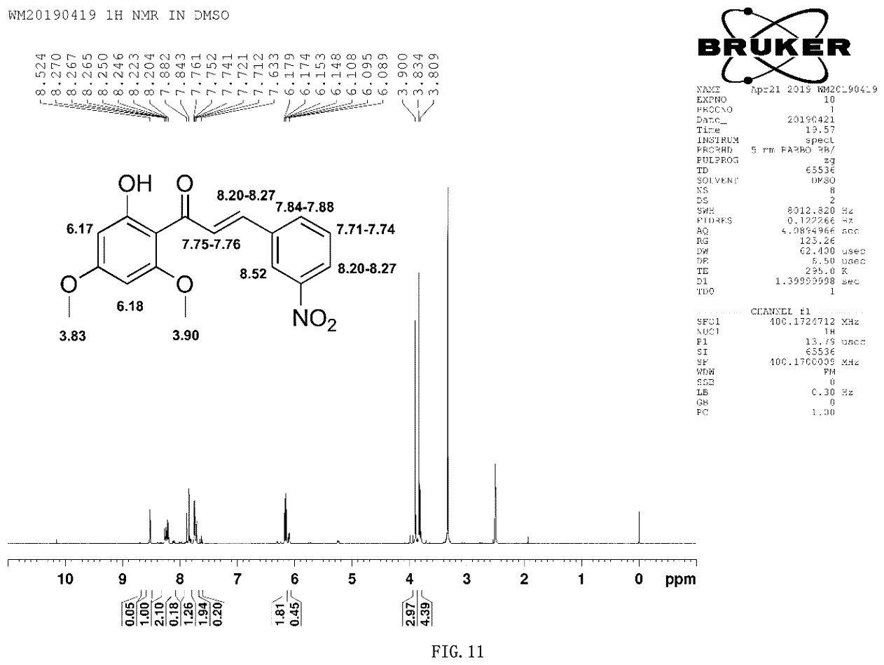

Identification was performed by an NMR method, and the results are shown in FIG. 11. The hydrogen spectrum data are as follows: 3.83 (s, 3H), 3.90 (s, 3H), 6.17-6.18 (d, 2H), 7.71-7.74 (t, 1H), 7.75-7.76 (d, 1H), 7.84-7.88 (d, 1H), 8.20-8.27 (m, 2H), and 8.52 (s, 1H), which meet the structural requirements of the molecular design.

2.5.4 Synthesis and Identification of Xn-4 Compound

2.5.4.1 Synthesis of Xn-4 Compound

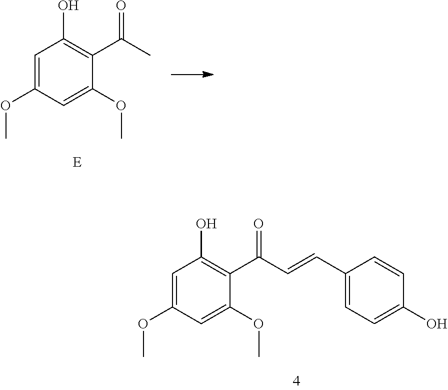 [SMILES: COc1cc(O)c(C(C)=O)c(OC)c1 ; COc1cc(O)c(C(=O)/C=C/c2ccc(O)cc2)c(OC)c1]

Chemical components: 10 g of compound E, 5.7 g of potassium hydroxide, 60 ml of methanol, and 7.5 g of p-hydroxybenzaldehyde.

Operation: 5.7 g of potassium hydroxide was added to 60 mL of methanol, the reaction solution was cooled to room temperature 25° C., and 10 g of compound E and 7.5 g of p-hydroxybenzaldehyde were added sequentially. The reaction was performed at room temperature for 24 h. After TLC detected that the reaction was complete, the reaction solution was added to 400 mL of water and filtered. The filter cake was taken out to reflux for 1 h by adding 100 mL of anhydrous ethanol, the reaction solution was cooled to 0° C., allowed to stand for 1 h and filtered, and the filter cake was dried to obtain about 13 g of product Xn-4.

2.5.4.2 Identification Results of Xn-4 Complex

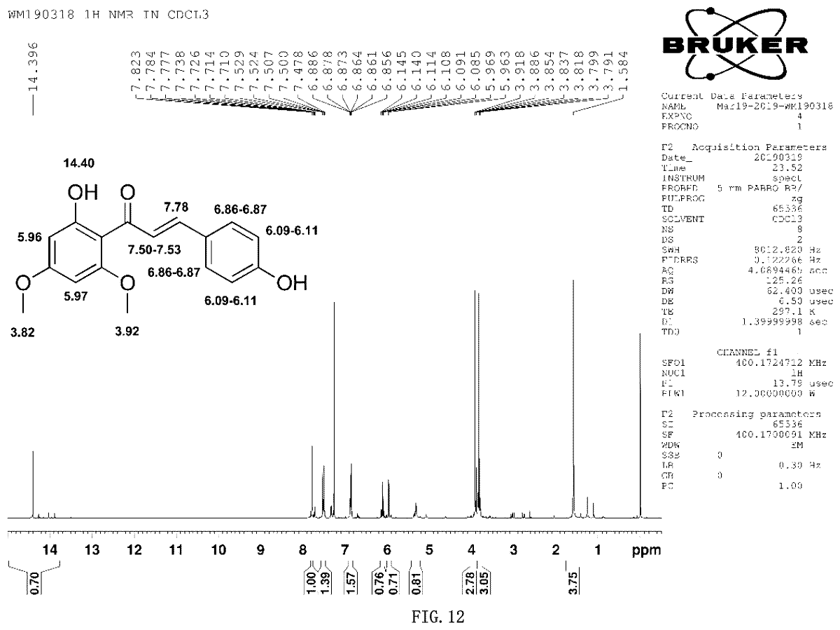

Identification was performed by an NMR method, and the results are shown in FIG. 12. The hydrogen spectrum data are as follows: 3.88 (s, 3H), 5.96 (s, 1H), 5.97 (s, 1H), 6.09-6.11 (d, 2H), 6.86-6.87 (d, 2H), 7.50-7.53 (d, 1H), 7.78 (d, 1H), and 14.40 (s, 1H), which meet the structural requirements of the molecular design.

2.5.5 Synthesis and Identification of Xn-5 Compound

2.5.5.1 Synthesis of Xn-5 Compound

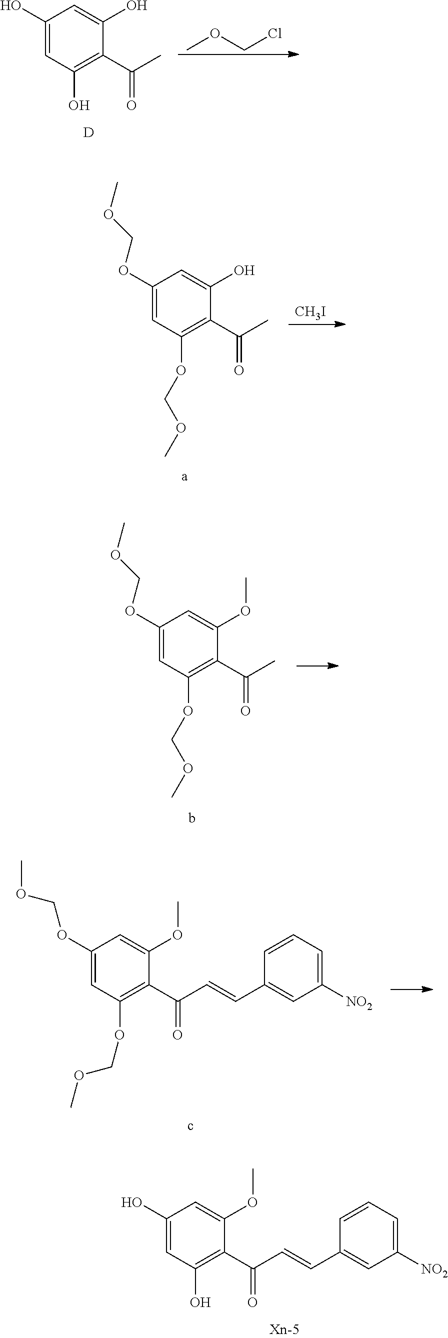

Step I:

Chemical components: 22 g of compound D, 20 g of MOMCl, 40 g of triethylamine, and 300 ml of dichloromethane.

Operation: Compound D and dichloromethane were added to a reaction flask sequentially, stirred evenly, and cooled down with brine ice. Triethylamine was added when the temperature in the flask reached 20° C., and stirring and cooling were continued. When the internal temperature reached 0° C., MOMCl was added dropwise. After dropwise addition, the temperature was allowed to rise to room temperature naturally. The reaction continued for 5 h, and then the reaction solution was poured into 1400 ml of water, stirred and separated. 500 ml of dichloromethane was added to the aqueous layer, and the reaction solution was mixed and separated. The organic layers were mixed and washed twice with 200 ml of water. The organic phase was concentrated and further separated and purified by column chromatography to obtain the product (compound a) (10 g).

Step II:

Chemical components: 2 g of compound a, 5.6 g of methyl iodide, 5 g of potassium carbonate, and 40 ml of DMF.

Operation: Compound a, methyl iodide, potassium carbonate and DMF were added sequentially into a reaction flask. After 4 h of reaction at 20° C., the reaction solution was poured into 500 mL of water. Then 200 mL of methylene chloride was added, stirred uniformly, and allowed to stand and separate. The organic layer was washed 3 times with 300 ml of water, dried and concentrated by suction filtration to obtain the product (compound b) (3 g).

Step III:

Chemical components: 3 g of compound b, 6 g of m-nitrobenzaldehyde, 12 g of potassium hydroxide, and 50 ml of ethanol.

Operation: Compound b, m-nitrobenzaldehyde, potassium hydroxide and ethanol were added sequentially into a reaction flask, stirred, and reacted at room temperature for 3 h. Then, the reaction solution was added to 500 mL of ice water, and extracted with 200 ml of dichloromethane, and the extract was washed once with 300 mL of water, dried and concentrated by suction filtration to obtain compound c (5 g).

Step IV:

Chemical components: 5 g of compound c, 20 ml of 10% hydrochloric acid, and 50 ml of methanol.

Operation: Compound c, methanol and hydrochloric acid were added to a reaction flask, and reacted at 50° C. for 40 min. The reaction solution was put into 500 mL of ice water, and extracted with 200 mL of ethyl acetate. The extract was washed with water once, dried and concentrated by suction filtration to obtain product Xn-5 (3 g).

2.5.5.2 Identification Results of Xn-5 Complex

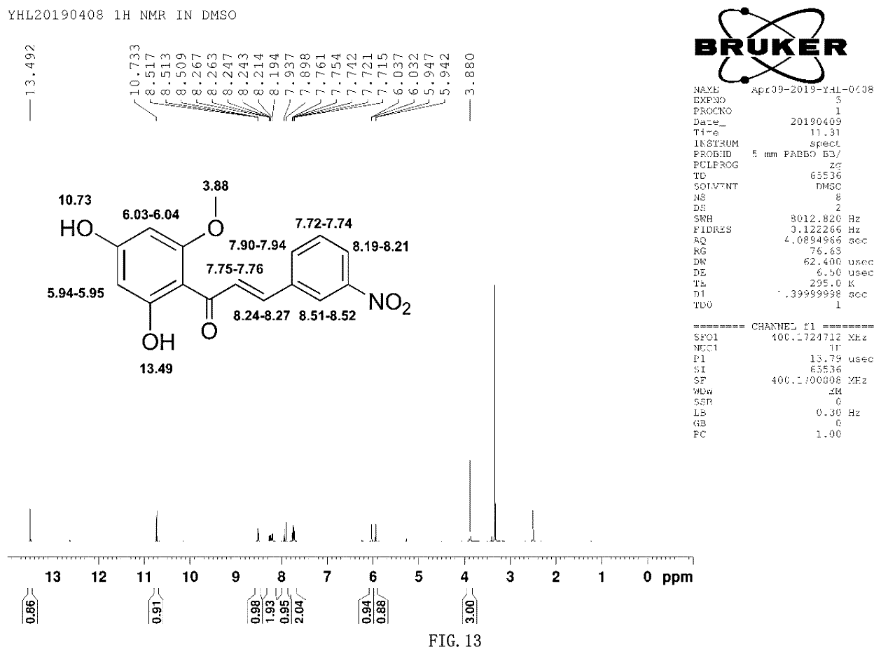

Identification was performed by an NMR method, and the results are shown in FIG. 13. The hydrogen spectrum data are as follows: 3.82 (s, 3H), 5.94-5.95 (s, 1H), 6.03-6.04 (s, 1H), 7.75-7.76 (d, 2H), 7.72-7.74 (t, 1H), 7.75-7.76 (d, 1H), 7.90-7.94 (d, 1H), 8.19-8.21 (d, 1H), 8.24-8.27 (d, 1H), 8.51-8.52 (s, 1H), 10.73 (s, 1H), and 13.49 (s, 1H), which meet the structural requirements of the molecular design.

2.6 Comparison of Inhibitory Effects of Different Derivatives of Xanthohumol on PRRSV Replication

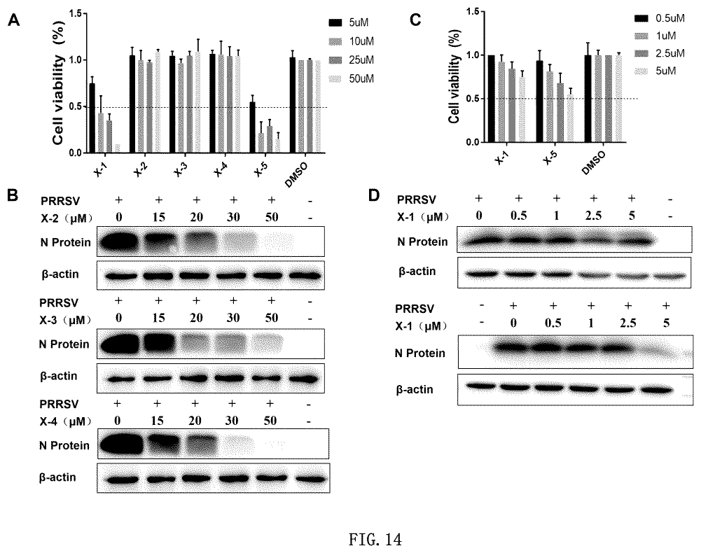

The cytotoxicity of the 5 synthetic compounds to Marc-145 cells was determined, and it was found that X-2, X-3 and X-4 were less cytotoxic and did not produce cytotoxicity within 50 uM (FIG. 14A). 0-50 uM X-2, X-3 and X-4 were used to determine their anti-PRRSV activity on Marc-145, and the results are shown in FIG. 14B. All the three compounds had inhibitory effects on PRRSV, and X-4 had the best effect, which had an obvious antiviral effect when the concentration was 15 μM, followed by compound X-2.

The cytotoxicity and antiviral ability experiments of X-1 and X-5 were carried out at low concentrations, and the results showed that X-1 and X-5 were highly cytotoxic and did not have antiviral ability (FIGS. 14C-14D).

2.7 Therapeutic Effects of Xanthohumol Derivative Xn-4 on PRRSV-Infected Piglets

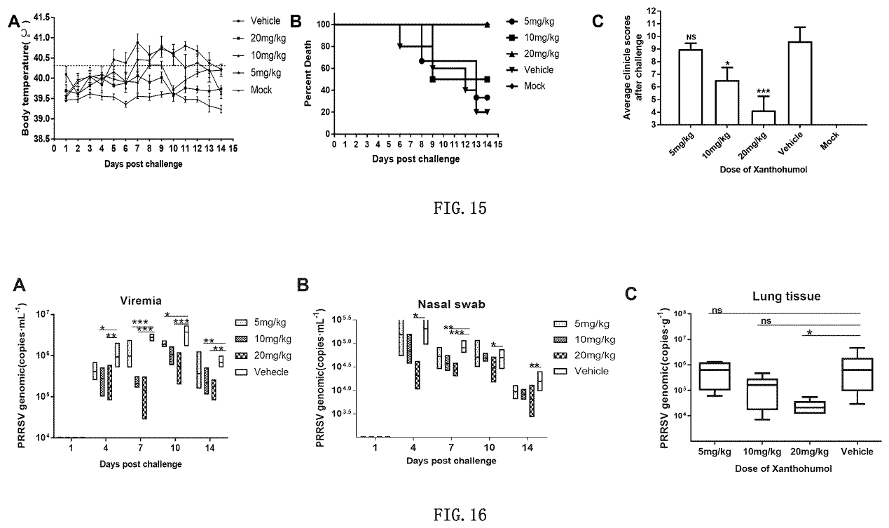

2.7.1 Clinical Symptoms During days 0-14 post virus infection, the rectal temperature of the piglets was measured (FIG. 15A), and the survival rate (FIG. 15B) and clinical symptoms (FIG. 15C) of the piglets were observed. The results were as follows: all piglets in the control group developed high fever (≥40.5° C.) post virus infection, showing clinical symptoms such as loss of appetite, lethargy, rough hair, dyspnea, periorbital edema, and mild diarrhea. 5-14 days post virus inoculation (dpi), 4 out of 5 pigs died one after another. The rectal temperature of the piglets in the treatment group with 10 mg/kg Xn-4 increased 14 days post infection, and 1 pig died at 10 dpi. The infected piglets and uninfected piglets in the treatment group with 20 mg/kg Xn-4 had no clinical fever or obvious clinical symptoms, and all were healthy and alive during the experiment.

2.7.2 Detection Results of Viremia, and Viral Loads of Lung Tissue and Nasal Swabs

Serum and nasal swab samples were collected at 1 dpi, 4 dpi, 7 dpi, 10 dpi, and 14 dpi respectively, and the copy number of PRRSV genome cDNA was determined by qRT-PCR. The results were as follows: the virus level in the serum of infected pigs treated with 10 mg/kg Xn-4 and 20 mg/kg Xn-4 was significantly lower than that of the virus-infected positive control group at 4-14 dpi (FIG. 16A) (P<0.05). The copy number of the PRRSV genome in the nasal swabs of piglets in the treatment group with 20 mg/kg Xn-4 was significantly lower than that in the virus-infected positive control group at 4-10 dpi (FIG. 16B). The piglets were killed and dissected at 14 dpi, and the viral load in the lungs of each piglet was detected. The results were as follows: the PRRSV viral load in the lungs of the piglets in the treatment group with 20 mg/kg Xn-4 was significantly lower than that in the control group (P<0.05) (FIG. 16C) (P<0.05).

2.7.3 Pathological Changes of Lung Tissue

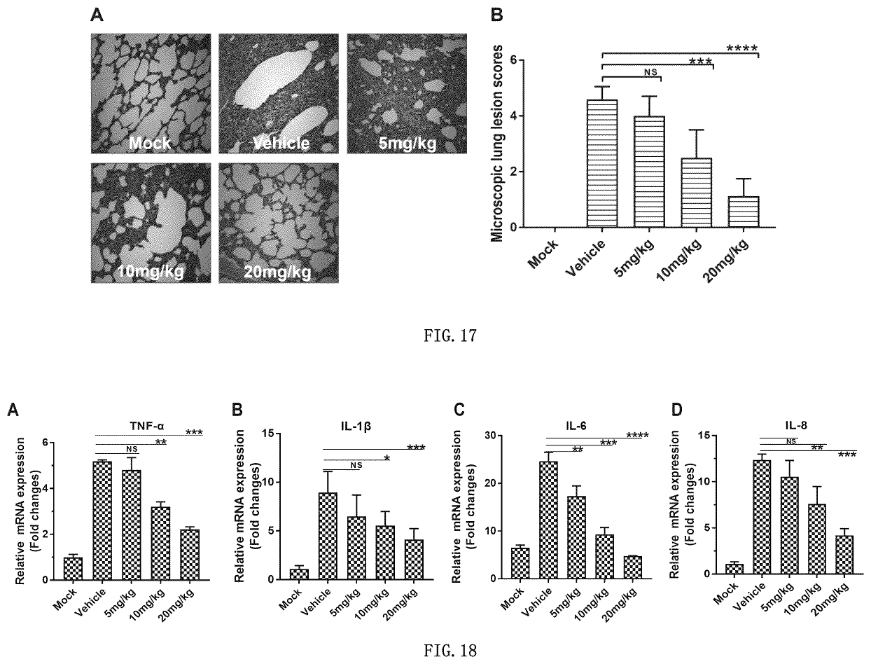

Histopathological observation showed that the lungs in the negative control group were normal, while the alveolar septa in the virus-positive control group thickened, with scattered hemorrhages in the alveolar septa, increased inflammatory cells, and inflammatory exudates in the trachea (FIG. 17A). The lung pathological change scores of the piglets treated with 10 mg/kg Xn-4 and 20 mg/kg Xn-4 were significantly lower than that of the virus-infected positive control group (P<0.05) (FIG. 17B).

2.7.4 Inflammatory Reaction of Lung Tissue

RNAs were extracted from the lung tissue of piglets in each group, and the mRNA levels of inflammatory factors IL-1β, IL-6, IL-8 and TNF-α in the lung tissue were detected by qRT-PCR. The results showed that the inflammatory factors in the lungs of the piglets in the positive control group increased significantly, while the inflammatory factors in the lung tissue of the piglets treated with Xn-4 decreased in a dose-dependent manner (FIG. 18A-D), indicating that Xn-4 could alleviate the surge of PRRSV-induced inflammatory factors in the lung.

3. Discussion

PRRSV can infect pigs of any age, mainly causing reproductive disorders such as abortion, premature birth, stillbirth, weak fetus and mummified fetus in pregnant sows, and respiratory symptoms in piglets and fattening pigs. PRRSV was first reported in the United States in 1987 and has since spread rapidly around the world. The American PRRSV (CH-la strain) was first isolated from suspected PRRS cases in China in 1996 by the Harbin Veterinary Research Institute of the Chinese Academy of Agricultural Sciences. In 2006-2007, highly pathogenic HP-PRRSV swept across all provinces in China, which brought huge losses to the pig industry. The World Organization for Animal Health lists PRRS as a Class B disease, and the Animal Epidemic Prevention Law of the People's Republic of China lists the classic PRRS as a Class II animal epidemic disease and highly pathogenic PRRS as a Class I animal epidemic disease.

At present, in China, in addition to measures for PRRSV prevention such as strengthening management and carrying out strict disinfection, vaccine immunization is the main measure to prevent PRRS, including mainly inactivated vaccines and attenuated vaccines. However, due to the characteristics such as easy mutation, multi-strain co-infection and persistent infection, vaccine immunization cannot completely control the spread of PRRSV. Moreover, so far there is no effective drug for PRRS. Therefore, the development of new effective drugs is very important for the prevention and treatment of PRRS, and the research on anti-PRRSV drugs has also become an important research topic in international veterinary medicine.

Many host antiviral factors, such as interferon-stimulated genes (ISGs), viperin, Mx2, 2′, 5′-oligoadenylate synthase 1 (OAS1), interferon-induced tetrapeptide repeat protein 3 (IFIT3), and CH25H, have been reported to have antiviral activity against PRRSV infection. In addition, some microRNAs, siRNA and shRNA have also been proved to inhibit PRRSV replication. However, the anti-PRRSV activity of these antiviral factors in the host has not been intensively studied. Some domestic scholars have found that some natural compounds can also antagonize PRRSV, for example, 12-deoxyphorbol 13-phenyl20, ouabain, bufalin, and valinomycin can inhibit the replication of PRRSV at micromolar concentrations; sodium tanshinone IIA sulfonate exerts anti-PRRSV activity by directly inactivating PRRSV and inhibiting the replication of PRRSV; procyanidin A2 has preventive and therapeutic effects on PRRSV; glycyrrhizic acid can inhibit the entry stage of PRRSV; and (−)-epigallocatechin-3-gallate also exhibits an anti-PRRSV replication effect. However, the antiviral mechanisms of these drugs and their antiviral effects in pigs have not been thoroughly studied.

In this study, xanthohumol (Xn) was screened from a drug library of 386 natural products. As an extract of hops, xanthohumol has a wide range of sources and mature extraction technology. Studies have reported that Xn has antiproliferative activity against breast, colon, and ovarian cancer cell lines. In our study, it was found that Xn inhibited PRRSV replication and PRRSV-induced inflammatory reaction in Marc-145 cells and PAM cells with a very low IC50 value, and showed the potential to treat PRRSV infection in PAM cells. Considering the high cost of purifying xanthohumol, 5 xanthohumol analogs were synthesized in this study, and it was found that the xanthohumol analog named “Xn-4” exhibited the optimal anti-PRRSV ability in in vitro experiments. In piglet treatment experiments, Xn-4 could alleviate PRRSV-induced clinical symptoms such as anorexia, fever (40-41° C.), lethargy, depression, and dyspnea, greatly reduce viremia, the viral load in lungs and intranasal excretion of infected piglets, and alleviate interstitial pneumonia induced by PRRSV, showing great potential as a PRRSV therapeutic drug. In this study, xanthohumol was discovered for the first time, and the analog Xn-4 thereof was synthesized artificially, which has clear anti-PRRSV pharmacological components, controllable quality, safety and non-toxicity, and has a good application prospect in the prevention and treatment of porcine reproductive and respiratory syndrome.

1. A method for preventing or treating porcine reproductive and respiratory syndrome comprising administering a drug comprising xanthohumol and/or a derivative thereof.

2. The method of claim 1, wherein the structure of the xanthohumol is shown in formula (I); 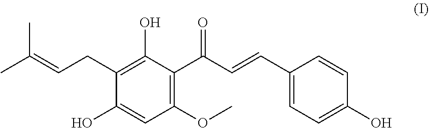 [SMILES: COc1cc(O)c(CC=C(C)C)c(O)c1C(=O)/C=C/c1ccc(O)cc1]
the structure of the xanthohumol derivative is shown in formula (II): 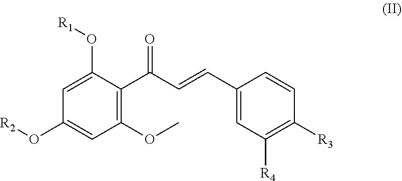
where R₁ is H or CH₃, R₂ is H or CH₃, R₃ is H or OH, and R₄ is H or NO₂.

3. A xanthohumol derivative, having a structure shown in formula (III):

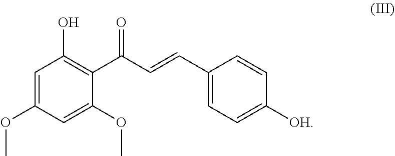 [SMILES: COc1cc(O)c(C(=O)/C=C/c2ccc(O)cc2)c(OC)c1]

4. A preparation method of the xanthohumol derivative of claim 3, comprising the following steps: 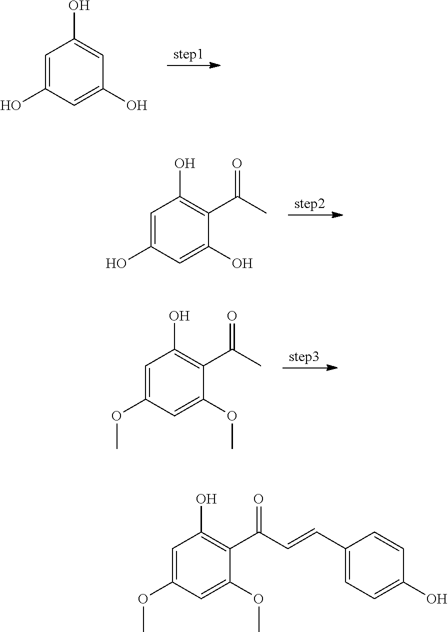 [SMILES: Oc1cc(O)cc(O)c1 ; CC(=O)c1c(O)cc(O)cc1O ; COc1cc(O)c(C(C)=O)c(OC)c1 ; COc1cc(O)c(C(=O)/C=C/c2ccc(O)cc2)c(OC)c1]
(1) adding m-trisphenol, isopropyl ether, acetonitrile and anhydrous zinc chloride into a reaction vessel, controlling the temperature at 0-5° C., and keeping stirring; introducing dry hydrogen chloride gas until the reaction solution becomes clear from turbidity; continuing to introduce the hydrogen chloride gas for producing a large amount of white solid; after gas introduction, immediately placing the reaction solution in a refrigerator at −10° C. overnight for crystallization; the next day, taking out and filtering the reaction solution, and washing the filter cake with isopropyl ether once; taking out the filter cake, adding water to fully disperse, starting heating, and carrying out atmospheric distillation to remove the residual isopropyl ether from the product; then continuing to heat up to 100° C. to reflux for producing a large amount of white solid, cooling the product to normal temperature, filtering the product, washing the filter cake with water and then drying the filter cake to obtain intermediate 1;
(2) adding the intermediate 1, acetone, anhydrous potassium carbonate and dimethyl sulfate into the reaction vessel, starting mechanical stirring, heating to reflux until the reaction is complete, filtering the reaction solution, washing the filter cake twice with acetone, concentrating the filtrate to obtain a solid, then washing the solid with petroleum ether and ethyl acetate (V:V) of 10:1, and carrying out filtering and drying to obtain intermediate 2; and
(3) adding potassium hydroxide to methanol, adding the intermediate 2 after cooling to room temperature, then adding p-hydroxybenzaldehyde, and stirring the mixture until the reaction is complete; adding the reaction solution to water, filtering the reaction solution, taking out the filter cake, adding absolute ethanol, heating the product to reflux, cooling the product to 0° C., and carrying out filtering and drying to obtain the target product.

5. A method for preventing or treating porcine reproductive and respiratory syndrome comprising administering a drug comprising the xanthohumol derivative of claim 3.

6. A drug for antagonizing porcine reproductive and respiratory syndrome virus, containing xanthohumol or the xanthohumol derivative of claim 3.
